(19) United States
(12) Patent Application Publication
(10) Pub. No.: US 2014/0018426 A1
(43) Pub. Date: Jan. 16, 2014

(54) FOOD OR BEVERAGE COMPOSITION FORTIFIED WITH THYRONAMINES AND/OR THYRONAMINE PRECURSORS

(71) Applicant: T*Amine, LLC, Salt Lake City, UT (US)

(72) Inventors: Joel R.L. Ehrenkranz, Salt Lake City, UT (US); Thomas S. Scanlan, Portland, OR (US)

(73) Assignee: T*Amine, LLC, Salt Lake City, UT (US)

(21) Appl. No.: 14/025,422

(22) Filed: Sep. 12, 2013

Related U.S. Application Data
(62) Division of application No. 12/878,514, filed on Sep. 9, 2010, now abandoned.
(60) Provisional application No. 61/241,639, filed on Sep. 11, 2009.

(51) Int. Cl. A61K 31/198

(52) U.S. Cl. CPC A61K 31/198
USPC 514/567

(57) ABSTRACT

Food or beverage compositions fortified with thyronines and/or thyronamines, processes for increasing T2, T1AM, and/or T0AM in a subject by administering a food or beverage composition that is fortified with at least one thyronine and/or thyronamine compound, and processes for promoting a healthy state by administering a food or beverage composition to a subject that is fortified with at least one thyronine and/or thyronamine compound. Increased levels of T2, T1AM, and/or T0AM in a subject can be associated with at least one of healthy cholesterol levels, healthy triglyceride levels, healthy blood sugar levels, cardiovascular health, healthy sleep patterns, healthy mood, healthy skin, healthy nails, or healthy endocrine function and/or a number of other markers of associated with general health and well-being.

### CROSS-REFERENCE TO RELATED APPLICATIONS

This application is a divisional of U.S. patent application Ser. No. 12/878,514, which was filed 9 Sep. 2010. U.S. patent application Ser. No. 12/878,514 claims the benefit of and priority to U.S. Provisional Patent Application Ser. No. 61/241,639 filed 11 Sep. 2009 entitled “FOOD OR BEVERAGE COMPOSITION FORTIFIED WITH THYRONAMINES AND/OR THYRONAMINE PRECURSORS.” The entireties of the above listed applications are incorporated herein by reference.

### BACKGROUND

Thyroid hormone is an important regulator of vertebrate development and homeostasis. Thyroid hormone is critical for normal fetal brain development, and brain disorders such as cretinism can result from a lack of thyroid hormone in the developing fetus. In adults, thyroid hormone exerts effects in almost all tissues, and important processes such as metabolic rate, thermal regulation, lipid inventory, cardiac function, and bone maintenance are affected by thyroid hormone. Individuals with excess blood levels of thyroid hormone (hyperthyroid) generally have elevated metabolic rate and body temperature, decreased serum cholesterol, and increased heart rate compared to those with normal thyroid hormone levels (euthyroid). Conversely, hypothyroidism is characterized by depressed metabolic rate and body temperature, elevated serum cholesterol, and decreased heart rate compared to euthyroid controls.

Thyronines are generally regarded as the principle chemical form of thyroid hormone. Thyronines include two phenyl ring structures joined by an oxygen. The general structure of a thyronine ((2S)-2-amino-3-[4-(4-hydroxy-3,5-(R)-phenoxy)-3,5-(R)-phenyl]propanoic acid) can be seen at Formula 1:

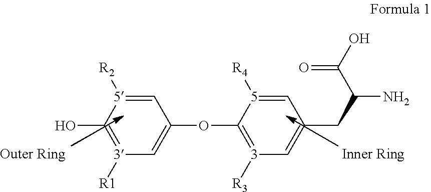

The two rings are referred to as the “inner ring” and the “outer ring.” As shown in Formula 1, the aminohydroxypropionic acid side chain on the inner ring includes a chiral center. Thyronines are produced in vivo by a series of enzymatically catalyzed reactions in the thyroid gland. Naturally occurring thyronines are typically derivatized with iodine at one or more of the 3 and 5 positions of the “inner ring” and the 3′ and 5′ positions of the “outer ring.”. Naturally occurring thyronines are of the/form; the chiral center has an absolute stereochemistry of S.

3,5,3′,5′-tetra-iodothyronine (“Thyroxine,” “T4,” or (2S)-2-amino-3-[4-(4-hydroxy-3,5-diiodophenoxy)-3,5-diiodophenyl]propanoic acid) is the predominant form of thyroid hormone that is secreted from the thyroid gland. T4 is synthesized by enzymes in the thyroid by joining the phenyl rings of two tyrosine residues and iodinating the two phenyl ring with a total of four iodine atoms at the R₁-R₄ positions of Formula 1. The structure of T4 is shown below:

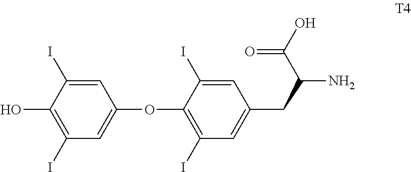

T4 is not the active form of thyroid hormone. Instead, T4 is converted to the physiologically active l-3,5,3′-triiodothyronine (“T3,” (2S)-2-amino-3-[4-(4-hydroxy-3-iodophenoxy)-3,5-diiodophenyl]propanoic acid) by enzymatic deiodination in peripheral target tissues. T3 is shown below:

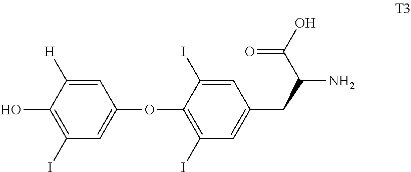

Three different deiodinases have been identified to date (D-I, D-II, and D-III). The D-I and D-II enzymes mediate “outer ring” deiodination such as the conversion of T4 to T3. In contrast, the D-III enzyme mediates “inner ring” deiodination, exemplified by the conversion of T4 to 3,3′,5′-triiodothyronine (“reverse-T3” or “rT3”). rT3 is shown below:

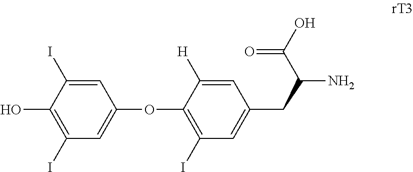

To date, no significant biological activity has been ascribed to rT3 even though significant blood levels of this metabolite are found. A variety of further deiodinated thyronines are known to exist in vivo. For example, T2 (“3,5-L-T2,” (2S)-2-amino-3-[4-(4-hydroxyphenoxy)-3,5-diiodophenyl]propanoic acid) may be made directly in the thyroid or it may be made by diodination of T4 or T3. T2 is shown below:

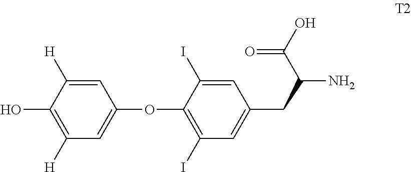

Another class of thyroid hormones known as thyronamines are thought to be produced by decarboxylation of thyronines. The enzymatic pathway responsible for decarboxylating thyronines is presently unknown, although it is postulated that the aromatic amino acid decarboxylase that normally produces dopamine and serotonin could also act on iodothyronines. It is also believed that decarboxylases in the stomach may be able to decarboxylate thyronines that are consumed as part of the diet in order to produce thyronamines. A general thyronamine can be described by Formula 2, shown below:

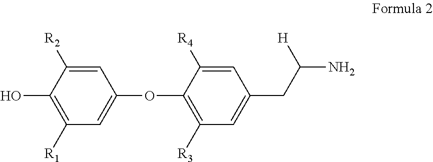

Thyronamines are similar to thyronines, except that the carboxyl group attached to the inner ring alkyl group is removed and replaced by a hydrogen. As shown in Formula 2, the chiral center is lost when a thyronine is decarboxylated to form a thyronamine. Thyronamines are similar to thyronines in that R₁-R₄ positions can be occupied by either iodine or hydrogen.

An example of a thyronamine is 3-iodothyronamine (“T1AM”), which is shown below at Formula 2A:

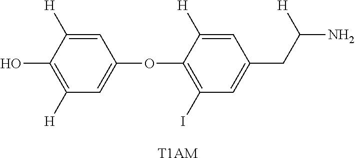

T1AM has been shown to be an endogenous component of biogenic amine extracts from rodent brain, liver, heart and blood.

### BRIEF SUMMARY

The illustrated embodiments relate to novel food or beverage compositions fortified with thyronines and/or thyronamines, processes for increasing 3,5-diiodo-L-thyronine (“T2,” “3,5-L-T2,” or “(2S)-2-amino-3-[4-(4-hydroxyphenoxy)-3,5-diiodophenyl]propanoic acid”), 3-iodothyronamine (“T1AM”) and/or thyronamine (“T0AM”) in a subject by administering a food or beverage composition that is fortified with at least one thyronine and/or thyronamine compound, and processes for promoting a healthy state by administering a food or beverage composition to a subject that is fortified with at least one thyronine and/or thyronamine compound. The illustrated embodiments are based partly on the discovery that intake of certain thyronines and/or thyronamines can increase levels of T2, T1AM, and/or T0AM in a subject. Such thyronines and/or thyronamines exhibit unexpectedly high bioavailability and they can be incorporated into a variety of food compositions. Increased levels of T2, T1AM, and/or T0AM in a subject can be associated with at least one of healthy cholesterol levels, healthy triglyceride levels, healthy blood sugar levels, cardiovascular health, healthy sleep patterns, healthy mood, healthy skin, healthy nails, or healthy endocrine function and/or a number of other markers of associated with general health and well-being.

In one embodiment, a food, beverage, or dietary supplement composition fortified with a thyronine and/or a thyronine or a stereoisomer, prodrug, pharmaceutically acceptable salt, hydrate, solvate, acid salt hydrate, N-oxide, or isomorphic crystalline salt thereof of Formula I is disclosed.

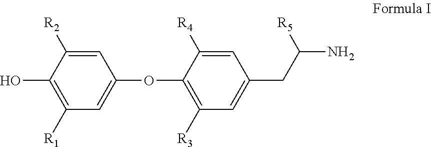

R₁-R₄ of Formula I are either I or H and R₅ of Formula I is either H or COOH. Essentially any combination of thyronines and/or thyronamines can be included in the fortified composition with the proviso that the compound of Formula I is not 3,5,3′,5′-tetra-iodothyronine (“T4”) or 3,3′,5-triiodothyronine (“T3”), or a prodrug, pharmaceutically acceptable salt, hydrate, solvate, acid salt hydrate, N-oxide, or isomorphic crystalline salt of T4 or T3.

Suitable examples of compounds of Formula I that can be included in the food, beverage, or dietary supplement composition described herein include T4AM, rT3, rT3AM, 3,3′-T2, 3,3′-T2AM, T2,3,5-T2AM, T1, T1AM, T0, and/or T0AM, and combinations thereof.

In one embodiment, the compound of Formula I included in the food, beverage, or dietary supplement composition described herein is T2 ((2S)-2-amino-3-[4-(4-hydroxyphenoxy)-3,5-diiodophenyl]propanoic acid) or a prodrug, pharmaceutically acceptable salt, hydrate, solvate, acid salt hydrate, N-oxide, or isomorphic crystalline salt thereof.

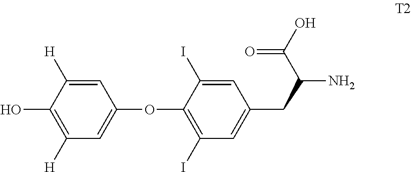

In some embodiments, a food, beverage, or dietary supplement composition supplemented with T2 may further include a compound selected from the group consisting of T4AM, rT3, rT3AM, 3,3′-T2, 3,3′-T2AM, 3,5-T2AM, T1, T1AM, T0, T0AM, and combinations thereof or a prodrug, pharmaceutically acceptable salt, hydrate, solvate, acid salt hydrate, N-oxide, or isomorphic crystalline salt thereof.

In another embodiment, the present invention includes a process for increasing T2, T1AM, and/or T0AM levels in a subject. The process includes (1) administering a fortified food, beverage, or dietary supplement composition that includes an effective amount of a compound of Formula I or a stereoisomer, prodrug, pharmaceutically acceptable salt, hydrate, solvate, acid salt hydrate, N-oxide, or isomorphic crystalline salt thereof to the subject, and (2) obtaining an effect of increasing T2, T1AM, and/or T0AM levels in the subject.

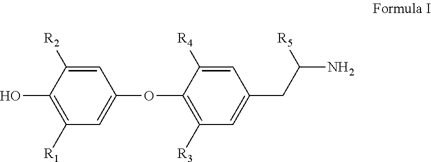

R₁-R₄ of Formula I are either I or H and R₅ of Formula I is either H or COOH. As above, the process described herein includes the proviso that the compound of Formula I is not 3,5,3′,5′-tetra-iodothyronine (“T4”) or 3,3′,5-triiodothyronine (“T3”), or a prodrug, pharmaceutically acceptable salt, hydrate, solvate, acid salt hydrate, N-oxide, or isomorphic crystalline salt of T4 or T3.

In yet another embodiment, a process for promoting at least one of healthy cholesterol levels, healthy triglyceride levels, healthy blood sugar levels, cardiovascular health, healthy sleep patterns, healthy mood, healthy skin, healthy nails, or healthy endocrine function and/or treating at least one of diabetes mellitus, fibromyalgia, sleep disorder, mood disorder, hyperglycemia, hypoglycemia, arthritis, physical or psychological condition caused by stress, or substance addiction in a human is disclosed. The process includes (1) administering to a human a daily dosage ranging from about 1 mcg to about 5000 mg of a compound of Formula I or a stereoisomer, prodrug, pharmaceutically acceptable salt, hydrate, solvate, acid salt hydrate, N-oxide, or isomorphic crystalline salt thereof, and

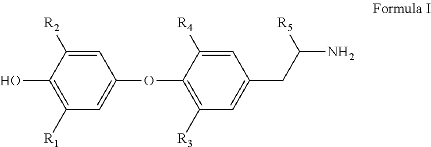

(2) obtaining an effect of promoting at least one of healthy cholesterol levels, healthy triglyceride levels, healthy blood sugar levels, cardiovascular health, healthy sleep patterns, healthy mood, healthy skin, healthy nails, or healthy endocrine function and/or treating at least one of diabetes mellitus, fibromyalgia, sleep disorder, mood disorder, hyperglycemia, hypoglycemia, arthritis, physical or psychological condition caused by stress, or substance addiction.

R₁-R₄ of Formula I are either I or H and R₅ of Formula I is either H or COOH. As above, the process described herein includes the proviso that the compound of Formula I is not 3,5,3′,5′-tetra-iodothyronine (“T4”) or 3,3′,5-triiodothyronine (“T3”), or a prodrug, pharmaceutically acceptable salt, hydrate, solvate, acid salt hydrate, N-oxide, or isomorphic crystalline salt of T4 or T3.

These and other objects and features of the present invention will become more fully apparent from the following description and appended claims, or may be learned by the practice of the invention as set forth hereinafter.

### DETAILED DESCRIPTION

### I. Introduction

Obesity, hyperlipidemia, hypercholesterolemia, and other unhealthy lifestyle choices represent major risk factors for diabetes, heart disease, stroke, and cancer. Interventions such as diet, exercise, surgical procedures, and medications which produce weight loss or lower cholesterol decrease the incidence of these major causes of morbidity and mortality. Compliance with lifestyle changes, such as diet and exercise, is very difficult to maintain, bariatric surgery is invasive, and medications which lower cholesterol are weight neutral and require a physician's prescription. 3,5-diiodo-L-thyronine (“T2,” “3,5-L-T2,” or “(2S)-2-amino-3-[4-(4-hydroxyphenoxy)-3,5-diiodophenyl]propanoic acid”), 3-iodothyronamine (“T1AM”), and/or thyronamine (“T0AM”) may represent novel agents with a unique mechanism of action for intervening in the pathophysiology of these disorders and lifestyle choices.

The illustrated embodiments relate to novel food or beverage compositions fortified with thyronines and/or thyronamines, processes for increasing T2, T1AM, and/or T0AM in a subject by administering a food or beverage composition that is fortified with at least one thyronine and/or thyronamine compound, and processes for promoting a healthy state by administering a food or beverage composition to a subject that is fortified with at least one thyronine and/or thyronamine compound. The illustrated embodiments are based partly on the discovery that intake of certain thyronines and/or thyronamines can increase levels of T2, T1AM, and/or T0AM in a subject. Such thyronines and/or thyronamines exhibit unexpectedly high bioavailability and they can be incorporated into a variety of food compositions. Increased levels of T2, T1AM, and/or T0AM in a subject can be associated with at least one of healthy cholesterol levels, healthy triglyceride levels, healthy blood sugar levels, cardiovascular health, healthy sleep patterns, healthy mood, healthy skin, healthy nails, or healthy endocrine function and/or a number of other markers of associated with general health and well-being.

Many known biological activities of thyroid hormone are mediated by binding of T3 to thyroid hormone receptors (TRs), although there are a number of known effects that are not mediated by TRs. T3 binds to the ligand binding domain (LBD) of nuclear localized TRs, and the activated TR regulates the transcription of hormone responsive genes. In this mode of action, the effects of thyroid hormone are manifested exclusively through positive and negative regulation of hormone-responsive gene transcription.

Recently, T1AM and T0AM have been shown to be endogenous components of biogenic amine extracts from rodent brain, liver, heart and blood. T1AM and T0AM are shown below:

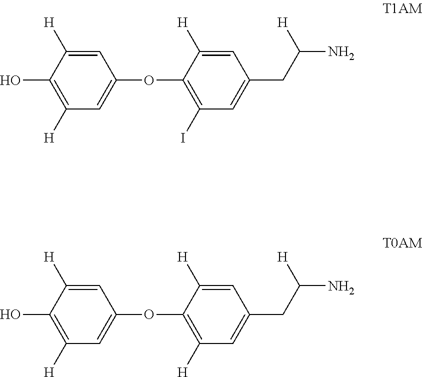 [SMILES: NCCc1ccc(Oc2ccc(O)cc2)c(I)c1 ; NCCc1ccc(Oc2ccc(O)cc2)cc1]

T1AM has been found to be a potent agonist of trace amine-associated receptor 1 (TAAR1), an orphan G protein-coupled receptor (GPCR) that is highly homologous to adrenergic, dopaminergic, and serotonergic GPCRs. In addition, T1AM is an agonist of alpha-2A adrenergic and serotonin 5HT-2c receptors, and binds and inhibits the transporter function of vesicular monoamine, dopamine, norepenephrin, and serotonin transporters. T1AM has also been found to rapidly and reversibly decrease core body temperature and heart rate. In mice, T1AM induced an approximate 10° C. drop in body temperature that reached a nadir about 1 hour after injection and returned to normal after 4-6 hours, depending on the T1AM dose. Similar results have been reported for T0AM, although T0AM appears to be less potent. The T1AM induced reduction in cardiac output was found to be a direct effect, and independent of the T1AM induced hypothermia. In a rat working heart preparation held at 37° C., introduction of T1AM into the perfusion buffer resulted in large and immediate decreases in both heart rate and systolic aortic pressure. Similar results have been reported for T0AM, although T0AM appears to be about 10-fold less potent as compared to T1AM.

These initial pharmacological findings indicate that single dose T1AM treatment can rapidly induce a hypo-metabolic state in rodents. The induction of hypothermia was thought to have potential neuroprotective benefit in the case of ischemic injury such as stroke. Indeed, T1AM treatment was found to reduce infarct volume by 40% in a middle cerebral artery occlusion (MCAO) stroke model in mice, and the degree of neuroprotection afforded by T1AM was found to correlate well with the magnitude of hypothermia that was induced by T1AM. Along with hypothermia and reduced cardiac function, single dose T1AM rapidly induces hyperglycemia in mice. For example, mice that receive an injection of T1AM show 2-5 fold increased blood glucose peaking 1 hour after the injection and returning to baseline after 3-4 hours. At present it is not certain whether this effect of T1AM is direct, or whether it is purely pharmacological or has physiological significance.

In addition, it has recently been discovered that a single dose T1AM can dramatically switch fuel utilization away from carbohydrates and toward lipids. Siberian hamsters (Phodopus sungorus), a hibernating rodent species, as well as mice, display a complete shift in respiratory quotient (RQ) from a normal, mixed carbohydrate and lipid value (0.90 for hamsters, 0.83 for mice) to an RQ value of approximately 0.7, indicating that a switch to pure lipid burning has occurred upon single dose T1AM administration. Interestingly, this 0.7 RQ persisted in summer acclimatized Siberian hamsters for at least 24 h after the T1AM injection, making this fueling shift the effect of longest duration yet observed.

The kinetics of this effect differs somewhat from the previously studied T1AM pharmacology. The onset of the RQ effect was slower than the onset of T1AM induced hypothermia, bradycardia, or hyperglycemia; these three effects reach a maximum about 1 hour after T1AM injection, whereas the complete RQ shift is reached about 4.5 hours post-injection. Consistent with the shift in RQ toward lipid utilization, T1AM treated Siberian hamsters had measurable urine ketone content that peaked 16 hours post-injection, again illustrating the extended duration of the T1AM induced lipid burning switch compared to hypothermia, bradycardia, or hypergycemia which typically return to baseline values within 4-6 hours after T1AM administration.

T1AM and T0AM appear to be a product of T4 metabolism and it is believed that administering precursor compounds (e.g., rT3, T2, and/or T1) can stimulate T1AM and/or T0AM levels. It is also known from animal studies that T1AM and/or T0AM administration can lead to decreased metabolic rate, hypothermia, bradycardia, hyperglycemia, and a number of other effects. By extension, it is believed T1AM and/or T0AM may be natural triggers for controlling metabolism and that administration of precursor compounds should produce these and other effect in humans. Supplying exogenous material (e.g., T1AM precursor compounds) as a component of a food composition could have beneficial effects on heat stress, anaerobic activity (e.g., increased ability to withstand hypoxia), increased oxygen demand, low-flow states (e.g., cardiogenic shock), weight loss, hyperlipidemia, seizures, tachycardia, asthma, tachypnea, respiratory failure, sepsis, and organ transplantation. Supplying exogenous T1AM or T0AM or precursor compounds may also be useful as a starvation mimetic.

T2 is a naturally occurring thyroid hormone known to affect hepatic and skeletal muscle oxidative metabolism and resting metabolic rate in euthyroid animals without affecting the pituitary thyroid axis. T2 is shown below:

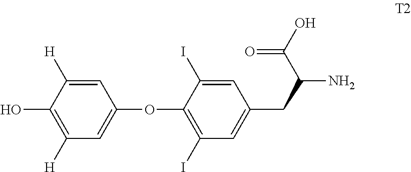

T2 appears to have the potential for safely and effectively mitigating the effects of increased carbohydrate and/or fat intake on normal human metabolic activity without disrupting endogenous thyroid function.

The embodiments illustrated herein are based partly on the surprising and unexpected discovery that intake of T2 is more effective than well-known statin drugs at lowering cholesterol in hypercholesterolemic rodents and that, even more surprisingly, T2 can decrease cholesterol via a mechanism that is independent of the low-density lipoprotein receptor (LDLr). In addition, the inventors have discovered that intake of T2 can lower blood sugar levels relative to controls, which suggests that T2 may be an effective diabetes treatment. Moreover, T2 may be combined with other thyroid hormones (e.g., T4 or T3), cholesterol lowering agents (e.g., statins), anti-diabetes agents, anti-hypertensivesypersensives, anti-coagulants, anti-anginals, anti-arrhythmics, and/or vitamin and mineral compositions in order to augment the effects of known and yet to be discovered therapeutics and to help maintain healthy triglyceride and cholesterol levels, healthy weight, cardiovascular health, healthy sleep patterns, healthy mood, healthy skin, healthy nails, healthy endocrine function, healthy metabolism, healthy neuropsychiatric function, and a number of other markers of associated with general health and well-being.

0.55% of the US population and approximately 10% of postmenopausal women are hypothyroid. Although the treatment of hypothyroidism with thyroxine is well accepted and generally effective in normalizing circulating TSH and free T4 levels, a significant number of hypothyroid patients receiving thyroxine report persistent subjective complaints despite standard therapy. The physiological basis for this incomplete treatment response is unclear. Adjuvant treatment of hypothyroidism with supplemental T3 has received attention, and the interest generated by research in this area demonstrates the widespread prevalence and clinical significance of this problem. Circulating T2 levels are reported to be low in hypothyroid patients and T4 and T3 are not thought to be converted to T2 in vivo. Anecdotal observations suggest that T2 has beneficial effects in myxedema (i.e., a disease resulting from the decreased function of the thyroid, characterized by a slowing down of mental and physical activity and thickening and drying of the skin). The role of T2 in the pathogenesis and treatment of hypothyroidism is an area which requires additional investigation.

Chronic metabolic abnormalities, including obesity, dyslipidemia, and hypothyroidism, are widespread. They represent the root cause of a number of diseases associated with substantial medical complications and health care expenses and are challenging to correct. New therapeutic strategies for treating obesity or lipid abnormalities have not been introduced in this decade nor have approaches for studying the chronic complaints commonly encountered in treated hypothyroidism been developed.

The first report showing a biological activity for T2 appeared in 1927. The effects of T2 are qualitatively distinct from those of T3. Specifically, T2 does not affect the pituitary thyroid axis, has selective effects on liver and brown fat, and demonstrates an onset of action, time to maximal effect, and duration of action significantly shorter than that of T3. T2's effects on mitochondrial energy production are not disrupted by protein synthesis inhibitors such as cyclophosphamide and actinomicin D, both of which completely block the mitochondrial effects of T3. In vitro studies show that T3 binds to TR α1, β1 and β2 with an affinity 40-500 fold greater than that of T2. T2, in contrast to T3, has no effect on thyroid receptor homodimer formation, is 50% less potent than T3 in decreasing TR β gene expression and 1/100 as potent as T3 in increasing growth hormone gene expression. T2 has <1% the potency of T3 in an in vivo anti-goiter assay and lacks central thyromimetic activity.

Immunoassay measurement of circulating total T2 levels in humans shows a concentration range of 0.4 to 10 ng/dl. There are no data on protein binding of T2 in serum. T2 levels decline with age and are higher in men than women. Women, however, produce more T2 and clear T2 more rapidly than men. T2 levels are increased in hyperthyroidism and decreased in hypothyroidism and sepsis.

A number of investigations from 1933 to the present have demonstrated T2's effects. For instance, T2 has been shown to have an effect on resting metabolic rate. The mechanism by which T2 increases metabolic rate is thought to be mediated by T2 acting on mitochondrial energy production. In vitro and in vivo studies have demonstrated that mitochondria have specific T2 binding sites. T2 increases mitochondrial cytochrome c oxidase, fatty acid and triacylglycerol synthesis, lipid oxidation, importing of fatty acids, F₀F₁ ATP synthase, and activates the AMPK-ACC-malonyl CoA pathway. In addition, chronic T2 administration to hypothyroid rats improves cold tolerance and normalizes somatic growth rates. In another study, it was found in rats that T2 administration (250 mcg/kg p.o. q.d. for 30 days) could decrease body weight by 13%, increase hepatic fat oxidation by 42%, decrease hepatic fat mass by 50%, and lower serum triglycerides by 52% and cholesterol by 18%, compared to controls. These metabolic effects were not accompanied by an increase in heart rate, altered thyroid gland or heart weight, changes in circulating TSH, free T3 or free T4 levels, or a blunted TSH response to TRH.

In another example, in 1960 McClure, de Mowbray, and Gilland administered a daily dose of 300 mg 3,5-D-T2 (i.e., the non-physiological stereoisomer of T2) for 8 months to 20 hypercholesterolemic patients, 13 of whom had coronary artery disease and 10 of whom had a history of myocardial infarction or angina. Of the remaining 20, 13 had no known atherosclerosis and 4 suffered from myxedema. McClure et al. observed a 5% decrease in body weight in euthyroid subjects and an 8% decrease in hypothyroid patients. Total serum cholesterol decreased by 20% at 20 weeks. An increase in mean heart rate from 76 to 88 beats per minute occurred. 7 of 13 patients with coronary artery disease experienced increased anginal symptoms. 2 of the 13 died suddenly from a presumed myocardial infarction. McClure et al. found that doses of less than 250 mg/day were ineffective and that patients receiving such a low dose had a tendency to “escape.” A daily dose of 300 mg/day T2 represents a 100 fold excess over T2 doses subsequently shown to have maximal effects on mitochondrial energy production.

As mentioned, it is believed that intake of certain thyronines and/or thyronamines can increase levels of T2, T1AM, and/or T0AM. In one embodiment, T2, T1AM, and/or T0AM can be administered directly to a subject. In another example, a subject can be administered one or more precursor compounds to increase levels of T2, T1AM, and/or T0AM. For example, it is possible that T2, T1AM, and/or T0AM can be produced in vivo from rT3 or another precursor by enzymatic deiodination and/or decarboxylation. Recent studies show that T1AM and higher-order iodinated thyronamines (e.g., T4AM, rT3AM, and 3,3′-T2AM) are subject to the similar metabolic processing as iodothyronines such as T4, suggesting a biological linkage between iodothyronines and iodothyronamines. The precise in vivo relationship between T1AM and thyronines is at present poorly understood; however, T1AM is clearly an endogenous chemical derivative of T4 and it is thus believed that T1AM and/or T0AM production can be stimulated in vivo by administration of precursors of T1AM and/or T0AM.

### II. Food, Beverage, or Dietary Supplement Compositions

In one embodiment, a food, beverage, or dietary supplement composition fortified with a thyronine and/or a thyronine or a stereoisomer, prodrug, pharmaceutically acceptable salt, hydrate, solvate, acid salt hydrate, N-oxide, or isomorphic crystalline salt thereof of Formula I is disclosed.

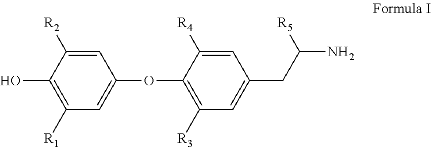

R₁-R₄ of Formula I are either I or H and R₅ of Formula I is either H or COOH. Essentially any combination of thyronines and/or thyronamines can be included in the fortified composition with the proviso that the compound of Formula I is not 3,5,3′,5′-tetra-iodothyronine (“T4”) or 3,3′,5-triiodothyronine (“T3”), or a prodrug, pharmaceutically acceptable salt, hydrate, solvate, acid salt hydrate, N-oxide, or isomorphic crystalline salt of T4 or T3.

Suitable examples of compounds of Formula I that can be included in the food, beverage, or dietary supplement composition described herein include T4AM, rT3, rT3AM, 3,3′-T2, 3,3′-T2AM, T2,3,5-T2AM, T1, T1AM, T0, and/or T0AM, and combinations thereof.

In the case of T4AM, R₁-R₄ of Formula I are I, and R₅ of Formula I is H. In the case of rT3 R₁, R₂, and R₃ of Formula I are I, R₄ of Formula I is H, and R₅ of Formula I is COOH. In the case of T3AM, R₁, R₃, and R₄ of Formula I are I, R₂ of Formula I is H, and R₅ of Formula I is H. In the case of rT3AM, R₁, R₂, and R₃ of Formula I are I, R₄ of Formula I is H, and R₅ of Formula I is H. In the case of 3,3′-T2, R₁ and R₃ of Formula I are I, R₂ and R₄ of Formula I are H, and R₅ of Formula I is COOH. In the case of 3,3′-T2AM, R₁ and R₃ of Formula I are I, R₂ and R₄ of Formula I are H, and R₅ of Formula I is H. In the case of T2, R₁ and R₂ of Formula I are H, R₃ and R₄ of Formula I are I, and R₅ of Formula I is COOH. In the case of T2AM, R₁ and R₂ of Formula I are H, R₃ and R₄ of Formula I are I, and R₅ of Formula I is H. In the case of T1, R₃ of Formula I is I, R₁, R₂, and R₄ of Formula I are H, and R₅ of Formula I is COOH. In the case of T1AM, R₃ of Formula I is I, R₁, R₂, and R₄ of Formula I are H, and R₅ of Formula I is H. In the case of T0, R₁-R₄ of Formula I are H, and R₅ of Formula I is COOH. In the case of T0AM, R₁-R₄ of Formula I are H, and R₅ of Formula I is H.

In a specific embodiment of the food, beverage, or dietary supplement composition disclosed herein, the compound of Formula I is T2 or a prodrug, pharmaceutically acceptable salt, hydrate, solvate, acid salt hydrate, N-oxide, or isomorphic crystalline salt thereof.

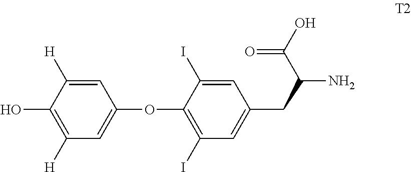

The T2 can be further combined with one or more compounds selected from the group consisting of T4AM, rT3, rT3AM, 3,3′-T2, 3,3′-T2AM, 3,5-T2AM, T1, T1AM, T0, T0AM or a prodrug, pharmaceutically acceptable salt, hydrate, solvate, acid salt hydrate, N-oxide, or isomorphic crystalline salt thereof.

In one embodiment, the food or beverage composition can be encapsulated in a plurality of vesicles that can be incorporated into the food or beverage composition. Vesicles can be configured according to the present invention such that they are substantially stable in the food or beverage medium while being degradable when the fortified food or beverage composition is consumed so as to deliver the compound of Formula I to the consumer.

Suitable examples of vesicles include, but are not limited to, liposomes, micelles, or reverse micelles. Generally the term liposome refers to a small (e.g., about 10 μm to about 100 μm), substantially spherical structure made out of a bilayer-forming material, having a head group that is attracted to water and a tail is repelled by water. Liposomes can be composed of naturally-derived phospholipids with mixed lipid chains (like egg phosphatidylethanolamine), or of pure surfactant components like DOPE (dioleoylphosphatidylethanolamine). Liposomes typically contain a small amount of aqueous solution inside the sphere. As such, liposomes can be used to encapsulate hydrophilic materials in the aqueous solution. Hydrophobic materials can also be dissolved and encapsulated in the lipid bilayer.

Micelles are very similar to liposomses except their shell consists of a single layer. In contrast to liposomes, micelles typically do not encapsulate aqueous material. Rather they can be used to stably encapsulate oils and other hydrophobic substances. Reverse micelles are similar to micelles except their orientation is reversed, with the hydrophilic heads pointed into the sphere and the hydrophobic tails pointed into the medium. Reverse micelles can be used to encapsulate and suspend hydrophilic substances in a hydrophobic medium.

In one embodiment, the plurality of vesicles can further include at least one decarboxylase. The vesicles can be configured such that the decarboxylase is included with the compound of Formula I or partitioned separately from the compound of Formula I. Including the decarboxylase can facilitate the conversion of thyronines to thyronamines.

The compounds of Formula I can be incorporated into a number of food and/or beverage compositions. Suitable examples of foods and/or beverages include, but are not limited to, processed meat products, processed fish products, gels such as energy gels, jams, pastes, nutrition bars, bakery products, creams, sauces, dairy products, confections, syrups, pet foods, water-based beverages, dairy-based beverages, complex carbohydrates, fats, proteins, prepared foods and beverages, dietary supplements, and combinations thereof.

Preferably, the fortified food or beverage compositions described herein include about 0.01 wt % to about 99.9 wt % of the compound of Formula I, or, more preferably, about 0.1 wt % to about 60 wt % of the compound of Formula I, or, most preferably, about 1 wt % to about 50 wt % of the compound of Formula I.

### III. Processes for Increasing T2, T1AM, and/or T0AM

In one embodiment, the present invention includes a process for increasing T1AM and/or T0AM levels in a subject. In another embodiment, the present invention includes a process for increasing T2, T1AM, and/or T0AM levels in a subject. The process includes (1) administering a fortified food, beverage, or dietary supplement composition that includes an effective amount of a compound of Formula I or a stereoisomer, prodrug, pharmaceutically acceptable salt, hydrate, solvate, acid salt hydrate, N-oxide, or isomorphic crystalline salt thereof to the subject, and (2) obtaining an effect of increasing T2, T1AM, and/or T0AM levels in the subject.

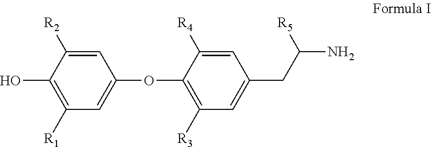

In one embodiment, R₁-R₄ of Formula I are either I or H, and R₅ of Formula I is either H or COOH. However, compounds of Formula I do not include 3,5,3′,5′-tetra-iodothyronine (“T4”) or 3,3′,5-triiodothyronine (“T3”), or a prodrug, pharmaceutically acceptable salt, hydrate, solvate, acid salt hydrate, N-oxide, or isomorphic crystalline salt of T4 or T3.

Suitable examples of compounds of Formula I that can be used in the method include T4AM, rT3, rT3AM, 3,3′-T2, 3,3′-T2AM, T2,3,5-T2AM, T1, T1AM, T0, T0AM, and combinations thereof.

In a specific embodiment, the compound of Formula I is T2 or a prodrug, pharmaceutically acceptable salt, hydrate, solvate, acid salt hydrate, N-oxide, or isomorphic crystalline salt thereof.

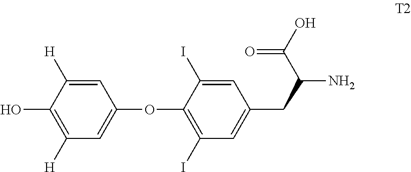

The process of claim 9, further comprising administering a compound selected from the group consisting of T4AM, rT3, rT3AM, 3,3′-T2, 3,3′-T2AM, 3,5-T2AM, T1, T1AM, T0, T0AM, and combinations thereof or a prodrug, pharmaceutically acceptable salt, hydrate, solvate, acid salt hydrate, N-oxide, or isomorphic crystalline salt thereof.

In one embodiment, the effective amount of the compound of Formula I includes a daily dosage of at least about 1 mcg, at least about 5 mcg, at least about 50 mcg, at least about 100 mcg, at least about 200 mcg, at least about 500 mcg, at least about 750 mcg, at least about 1 mg, at least about 10 mg, at least about 50 mg, at least about 100 mg, at least about 500 mg, at least about 1000 mg, at least about 2000 mg, at least about 5000 mg, or at least about 10,000 mg.

In another embodiment, the effective amount of the compound of Formula I includes a daily dosage of between about 1 mcg and about 10,000 mg, or between about 1 mcg and about 5000 mg, or between about 5 mcg and about 5000 mg, or between about 1 mg and about 2000 mg, or, preferably, a daily dosage of between about 10 mg and about 1000 mg, or, more preferably, a daily dosage of between about 50 mg and about 500 mg.

In one embodiment, the effective amount of the compound of Formula I is effective for promoting healthy cholesterol levels, healthy triglyceride levels, healthy blood sugar levels, cardiovascular health, healthy sleep patterns, healthy mood, healthy skin, healthy nails, or healthy endocrine function and/or treating at least one of diabetes mellitus, fibromyalgia, sleep disorder, mood disorder, hyperglycemia, hypoglycemia, arthritis, physical or psychological condition caused by stress, or substance addiction in a human.

In another embodiment, a process for promoting at least one of healthy cholesterol levels, healthy triglyceride levels, healthy blood sugar levels, cardiovascular health, healthy sleep patterns, healthy mood, healthy skin, healthy nails, or healthy endocrine function and/or treating at least one of diabetes mellitus, fibromyalgia, sleep disorder, mood disorder, hyperglycemia, hypoglycemia, arthritis, physical or psychological condition caused by stress, or substance addiction is disclosed. The method includes (1) administering to a human a daily dosage ranging from about 1 mcg to about 5000 mg of a compound of Formula I or a stereoisomer, prodrug, pharmaceutically acceptable salt, hydrate, solvate, acid salt hydrate, N-oxide, or isomorphic crystalline salt thereof; and

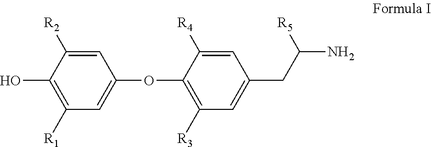

(2) obtaining an effect of promoting at least one of healthy cholesterol levels, healthy triglyceride levels, healthy blood sugar levels, cardiovascular health, healthy sleep patterns, healthy mood, healthy skin, healthy nails, or healthy endocrine function and/or treating at least one of diabetes mellitus, fibromyalgia, sleep disorder, mood disorder, hyperglycemia, hypoglycemia, arthritis, physical or psychological condition caused by stress, or substance addiction.

In one embodiment, R₁-R₄ are either I or H and R₅ is either H or COOH. However, the compound of Formula I is not 3,5,3′,5′-tetra-iodothyronine (“T4”) or 3,3′,5-triiodothyronine (“T3”), or a prodrug, pharmaceutically acceptable salt, hydrate, solvate, acid salt hydrate, N-oxide, or isomorphic crystalline salt of T4 or T3.

In a specific embodiment, the compound of Formula I is T2 or a prodrug, pharmaceutically acceptable salt, hydrate, solvate, acid salt hydrate, N-oxide, or isomorphic crystalline salt thereof.

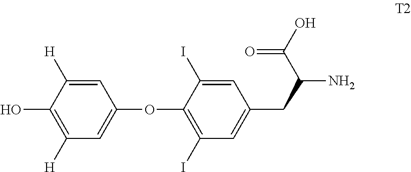

In one embodiment, the effective amount of the compound of Formula I includes a daily dosage of at least about 1 mcg, at least about 5 mcg, at least about 50 mcg, at least about 100 mcg, at least about 200 mcg, at least about 500 mcg, at least about 750 mcg, at least about 1 mg, at least about 10 mg, at least about 50 mg, at least about 100 mg, at least about 500 mg, at least about 1000 mg, at least about 2000 mg, at least about 5000 mg, or at least about 10,000 mg.

In another embodiment, the effective amount of the compound of Formula I includes a daily dosage of between about 1 mcg and about 10,000 mg, or between about 5 mcg and about 5000 mg, or between about 1 mg and about 2000 mg, or, preferably, a daily dosage of between about 10 mg and about 1000 mg, or, more preferably, a daily dosage of between about 50 mg and about 500 mg.

In one embodiment, the effective amount of the compound of Formula I is administered in a fortified food or beverage composition. Suitable examples of foods and/or beverages include, but are not limited to, processed meat products, processed fish products, gels such as energy gels, jams, pastes, nutrition bars, bakery products, creams, sauces, dairy products, confections, or syrups, pet foods, water-based beverages, dairy-based beverages, fruits and vegetables, dietary supplements, and combinations thereof.

In one embodiment, the fortified food or beverage composition includes about 0.01 wt % to about 99.9 wt % of the compound of Formula I, or, preferably, about 0.1 wt % to about 60 wt % of the compound of Formula I, or, more preferably, about 1 wt % to about 50 wt % of the compound of Formula I.

### IV. Examples

Obesity, hyperlipidemia, hypercholesterolemia, and other unhealthy lifestyle choices represent major risk factors for diabetes, heart disease, stroke, and cancer. The inventors in the present case have found that administration of T2 is sufficient to lower serum cholesterol. Surprisingly and unexpectedly, the inventors have found that T2 is capable of lowering cholesterol in subjects that are non-responsive to statin treatment. In addition, the inventors have found that T2 administration is sufficient to lower blood sugar. The compositions and methods disclosed herein for increasing levels of T2, T1AM, and/or T0AM present novel approaches to address and intervene in the pathophysiology of these disorders and lifestyle choices.

Familial hypercholesterolemia (“FH”) is a genetic disorder characterized by high cholesterol levels, specifically very high LDL levels in the blood and early onset of cardiovascular disease. Type II diabetes is associated with obesity, hyperlipidemia, hypercholesterolemia, and other unhealthy lifestyle choices and is also associated with early onset of cardiovascular disease and other disorders of the circulatory system.

Most cases of familial hypercholesterolemia are associated with mutations in the LDLr gene; mutations in other genes are rare. Patients who have one abnormal copy (i.e., heterozygotes) of the LDLr gene may have premature cardiovascular disease at the age of 30 to 40. Having two abnormal copies (i.e., homozygotes) may cause severe cardiovascular disease in childhood. Risk of cardiovascular disease is increased further with age and in those who smoke, are overweight or obese, have diabetes, and/or high blood pressure. Heterozygous FH is a common genetic disorder, occurring in 1:500 people in most countries; homozygous FH is much rarer, occurring in about 1 in a million births.

Heterozygous FH is normally treated with statins, bile acid sequestrants or other hypolipidemic agents that lower cholesterol levels. Individuals having less than a threshold level of LDL receptor function (e.g., less than about 10%) do not respond to statins or other currently used cholesterol lowering treatments and may require other treatments, including LDL apheresis (removal of LDL in a method similar to dialysis) and occasionally liver transplantation.

Presented below are data illustrating the effect of low and high dose T2 administration on wild-type and mutant mice. Low dose animals received a daily oral dosage of T2 of 0.25 mg/kg and high dose animals received a daily oral dosage of T2 of 2.5 mg/kg.

Mice in the study were fed a standard lab chow referred to as the “Western Diet.” The overall level of fat and the saturated nature of the fat are representative diets typical in the industrialized west that are linked to risk of cardiovascular disease in humans. The formula is used primarily with genetically manipulated rodent models that are susceptible to high cholesterol and cardiovascular disease. The diet may also be useful in diet-induced obesity, diabetes, and metabolic syndrome models. High cholesterol, obesity, and diabetes are each associated increased risk for cardiovascular disease. The composition of the Western Diet is shown below in Table 1.

TABLE 1
Western Diet

| Component | g/kg |
|---|---|
| Casein | 195.0 |
| DL-Methionine | 3.0 |
| Sucrose | 341.46 |
| Corn Starch | 150.0 |
| Anhydrous Milkfat | 210.0 |
| Cholesterol | 1.5 |
| Cellulose | 50.0 |
| Mineral Mix, AIN-76 (170915) | 35.0 |
| Calcium Carbonate | 4.0 |
| Vitamin Mix, Teklad (40060) | 10.0 |
| Ethoxyquin, antioxidant | 0.04 |

C57 is a wild-type strain, LDLr+/− are a strain carrying one functional copy of the LDLr gene, and the LDLrO animals are total LDLr knockouts. The LDLr gene encodes for the LDL receptor protein, which is responsible for scavenging LDL with bound cholesterol from the blood and transporting it into cells. Both heterozygous and homozygous LDLr knockout animals develop very high serum cholesterol levels and are considered to be a good model for heterozygous and homozygous familial hypercholesterolemia in humans.

In the data presented below, low and high dose T2 administration did not affect serum triglyceride levels (Table 2) or weight gain (Tables 3 and 4) in any appreciable way. In contrast, both low and high doses of T2 lowered serum cholesterol levels in all animals (Table 5). Low dose T2 administration lowered cholesterol an average of about 38% in C57 animals and about 26% in LDLr+/− animals. High dose T2 administration lowered cholesterol an average of about 67% in C57 and LDLr+/− animals. Surprisingly, T2 administration was able to lower serum cholesterol levels in LDLr knockout animals—low and high dose T2 administration was able to lower cholesterol by an average of about 46% and about 80%, respectively. Cholesterol levels in the LDLr knockout animals were still relativelty high following T2 treatment, but the fact that T2 was able to lower cholesterol at all is surprising and unexpected given that statin treatment is known to be ineffective in individuals that have no or low levels of LDL receptor function. LDLr knockout animals have zero LDL receptor function. In addition, high doses of T2 were able to lower serum glucose levels in all animals as compared to controls (Table 6). High doses of T2 were able to lower blood sugar levels in all genetic groups by an average of about 45-50%. This effect was independent of weight loss.

TABLE 2
Triglycerides

| | Con | T2 | Con | T2 | Vehicle | T2 | Vehicle | T2 |
|---|---|---|---|---|---|---|---|---|
| | Before Diet | | 1 Week Diet | | 1 Week Low Dose | | 1 Week High Dose | |
| C57 | 51.371 | 83.487 | 53.403 | 224.600 | 19.997 | 13.299 | 20.735 | 45.486 |
| C57 | 69.328 | 134.597 | 183.750 | 228.000 | 34.882 | 36.371 | 43.269 | 50.657 |
| C57 | 60.695 | 72.436 | 217.790 | 54.465 | 32.277 | 43.441 | 53.982 | 72.822 |
| C57 | 77.616 | 68.292 | 173.530 | 176.940 | — | 24.835 | — | 42.161 |
| C57 | 75.544 | 76.235 | 180.340 | 200.770 | 36.371 | — | 41.791 | — |
| C57 | 68.292 | 67.947 | 132.680 | 193.960 | 18.509 | 29.673 | 48.441 | 68.02 |
| Ave | 67.1 | 83.8 | 156.9 | 179.8 | 28.4 | 29.5 | 41.6 | 55.8 |
| Stdev | 9.76 | 25.53 | 57.54 | 64.32 | 8.50 | 11.47 | 12.63 | 13.77 |
| P-value | | | | | | 0.865478 | | 0.127976 |
| LDLr0 | 80.379 | 91.43 | 388.010 | 428.860 | 178.892 | — | 214.677 | — |
| LDLr0 | 100.754 | 94.883 | 296.090 | 313.110 | 227.639 | 230.244 | 162.959 | 173.672 |
| LDLr0 | 81.76 | 93.847 | 394.810 | 377.790 | 189.683 | 106.329 | 167.392 | — |
| LDLr0 | | 79.688 | | 323.320 | | 212.382 | | 210.614 |
| Ave | 87.6 | 90.0 | 359.6 | 360.8 | 198.7 | 183.0 | 181.7 | 192.1 |
| Stdev | 11.39 | 7.00 | 55.14 | 53.54 | 25.60 | 66.98 | 28.67 | 26.12 |
| P-value | | | | | | 0.722927 | | 0.708139 |
| LDLr+/− | 62.767 | 79.688 | 166.720 | 159.920 | 13.671 | 12.927 | 36.25 | 40.683 |
| LDLr+/− | 62.422 | 60.004 | 146.300 | 187.150 | 28.556 | 34.138 | 28.862 | 35.511 |
| LDLr+/− | 63.112 | 71.746 | 146.300 | 170.130 | 25.951 | 40.464 | 38.097 | 62.848 |
| LDLr+/− | 81.76 | 65.875 | 228.000 | 139.490 | 21.486 | 29.673 | 41.791 | 68.389 |
| LDLr+/− | 82.451 | 53.443 | 245.020 | 125.870 | 30.045 | 31.161 | 78.733 | 37.359 |
| LDLr+/− | 64.839 | 80.379 | 47.031 | 210.980 | — | 40.092 | — | 68.02 |
| Ave | 69.6 | 68.5 | 163.2 | 165.6 | 23.9 | 31.4 | 44.7 | 52.1 |
| Stdev | 9.76 | 10.79 | 70.67 | 31.10 | 6.60 | 10.09 | 19.57 | 15.86 |
| P-value | | | | | | 0.260471 | | 0.164619 |
| | Before Diet | | 1 Week Diet | | 1 Week Low Dose | | 2 Week Low Dose | |
| LDLr0 | 83.515 | 54.956 | 252.938 | 217.657 | 288.810 | 183.413 | 239.255 | 225.895 |
| LDLr0 | 80.990 | 83.515 | 263.964 | 282.235 | 384.370 | 260.001 | 327.764 | 274.993 |
| LDLr0 | 67.779 | 82.350 | 264.279 | 261.759 | 270.190 | 239.273 | 302.381 | 402.246 |
| LDLr0 | 141.216 | 86.041 | 321.926 | 226.792 | 304.620 | 268.082 | 372.520 | 281.673 |
| LDLr0 | 111.103 | 99.058 | 272.154 | 281.920 | 345.373 | 244.895 | 418.945 | 462.031 |
| LDLr0 | 64.864 | 127.811 | 366.028 | 265.224 | 222.059 | 177.441 | 369.848 | 223.557 |
| LDLr0 | 89.732 | 83.904 | 325.076 | 276.249 | 317.267 | 263.163 | 490.421 | 485.745 |
| LDLr0 | | 77.881 | | 277.824 | | 252.975 | | 496.099 |
| Ave | 91.3 | 86.9 | 295.2 | 261.2 | 304.7 | 236.2 | 360.2 | 356.5 |
| Stdev | 26.7869 | 20.5814 | 42.5685 | 25.2729 | 52.3331 | 35.6881 | 81.31847 | 117.3491 |
| P-value | | | | | | 0.010278 | | 0.946355 |

TABLE 3
Body weight of Mice on Low Dose T2

| Genotype | Treatment | Baseline | 1 Wk Diet | Day 1 | Day 2 | Day 3 | Day 4 | Day 5 | Day 6 | Day 7 |
|---|---|---|---|---|---|---|---|---|---|---|
| C57 | Vehicle | 28.0 | 31.0 | 30.5 | 31.1 | 30.8 | 29.5 | 30.4 | 30.9 | 31.9 |
| C57 | Vehicle | 26.0 | 31.0 | 29.8 | 30.3 | 30.6 | 29.8 | 30.5 | 30.8 | 31.5 |
| C57 | Vehicle | 25.0 | 28.0 | 27.7 | 29.3 | 29.6 | 28.4 | 29.2 | 29.8 | 29.8 |
| C57 | Vehicle | 25.0 | 28.0 | 27.1 | 28.4 | 28.5 | 27.8 | 26.0 | — | — |
| C57 | Vehicle | 25.0 | 28.0 | 26.1 | 27.2 | 27.9 | 26.4 | 26.9 | 26.7 | 26.7 |
| C57 | Vehicle | 23.0 | 25.0 | 23.8 | 25.5 | 25.4 | 24.9 | 24.9 | 24.4 | 24.4 |
| | Ave | 25.3 | 28.5 | 27.5 | 28.6 | 28.8 | 27.8 | 28.0 | 28.5 | 28.9 |
| | Stdev | 1.63 | 2.26 | 2.46 | 2.06 | 2.02 | 1.88 | 2.38 | 2.86 | 3.23 |
| C57 | T2 | 26.0 | 27.0 | 26.5 | 28.2 | 28.3 | 26.8 | 27.3 | 28.1 | 29.5 |
| C57 | T2 | 24.0 | 28.0 | 27.0 | 27.7 | 27.8 | 27.2 | 28.3 | 29.9 | 30.7 |
| C57 | T2 | 23.0 | 24.0 | 23.0 | 24.1 | 23.3 | 23.7 | 24.5 | 24.8 | 26.2 |
| C57 | T2 | 24.0 | 28.0 | 26.4 | 26.3 | 27.9 | 26.7 | 27.5 | 28.7 | 29.0 |
| C57 | T2 | 24.0 | 24.0 | 24.0 | 25.5 | 26.2 | 25.1 | — | — | — |
| C57 | T2 | 23.0 | 26.0 | 24.1 | 24.9 | 25.6 | 25.4 | 26.4 | 26.1 | 26.1 |
| | Ave | 24.0 | 26.2 | 25.2 | 26.1 | 26.5 | 25.8 | 26.8 | 27.5 | 28.3 |
| | Stdev | 1.10 | 1.83 | 1.66 | 1.60 | 1.90 | 1.33 | 1.45 | 2.05 | 2.06 |
| LDLr0 | Vehicle | 28.0 | 30.0 | 30.0 | 31.3 | 31.2 | 30.3 | 31.2 | 31.5 | 31.7 |
| LDLr0 | Vehicle | 26.0 | 28.0 | 26.6 | 28.1 | 28.5 | 27.8 | 28.8 | 29.4 | 29.4 |
| LDLr0 | Vehicle | 23.0 | 25.0 | 24.3 | 24.9 | 26.4 | 24.7 | 24.5 | 24.6 | 24.8 |
| | Ave | 25.7 | 27.7 | 27.0 | 28.1 | 28.7 | 27.6 | 28.2 | 28.5 | 28.6 |
| | Stdev | 2.52 | 2.52 | 2.87 | 3.20 | 2.41 | 2.81 | 3.39 | 3.54 | 3.51 |
| LDLr0 | T2 | 22.0 | 24.0 | 23.1 | — | — | — | — | — | — |
| LDLr0 | T2 | 20.0 | 23.0 | 21.1 | 21.6 | 22.3 | 21.2 | 22.0 | 22.1 | 22.4 |
| LDLr0 | T2 | 22.0 | 23.0 | 22.7 | 23.6 | 24.4 | 22.9 | 22.2 | 21.7 | 20.6 |
| LDLr0 | T2 | 21.0 | 23.0 | 22.1 | 23.2 | 23.5 | 22.8 | 23.0 | 23.1 | 23.6 |
| | Ave | 21.3 | 23.3 | 22.3 | 22.8 | 23.4 | 22.3 | 22.4 | 22.3 | 22.2 |
| | Stdev | 0.96 | 0.50 | 0.87 | 1.06 | 1.05 | 0.95 | 0.53 | 0.72 | 1.51 |
| LDLr+/− | Vehicle | 23.0 | 24.0 | 23.3 | 245 | 24.7 | 23.5 | 23.9 | 24.4 | 25.2 |
| LDLr+/− | Vehicle | 24.0 | 25.0 | 25.2 | 25.9 | 26.4 | 25.1 | 25.7 | 26.1 | 27.3 |
| LDLr+/− | Vehicle | 24.0 | 26.0 | 25.4 | 25.7 | 26.3 | 25.5 | 26.7 | 27.5 | 28.2 |
| LDLr+/− | Vehicle | 25.0 | 26.0 | 26.1 | 273 | 27.8 | 26.9 | 27.6 | 27.0 | 27.1 |
| LDLr+/− | Vehicle | 28.0 | 32.0 | 31.3 | 32.9 | 32.4 | 30.8 | 31.6 | 31.5 | 32.1 |
| LDLr+/− | Vehicle | 27.0 | 31.0 | 29.0 | 28.4 | 26.9 | — | — | — | — |
| | Ave | 25.2 | 27.3 | 26.7 | 27.5 | 27.4 | 26.4 | 27.1 | 27.3 | 28.0 |
| | Stdev | 1.94 | 3.33 | 2.91 | 2.99 | 2.64 | 2.76 | 2.87 | 2.63 | 2.55 |
| LDLr+/− | T2 | 27.0 | 28.0 | 28.2 | 29.4 | 29.7 | 28.8 | 29.5 | 30.7 | 31.0 |
| LDLr+/− | T2 | 27.0 | 29.0 | 29.0 | 30.6 | 31.4 | 30.3 | 31.3 | 32.4 | 32.7 |
| LDLr+/− | T2 | 23.0 | 25.0 | 24.7 | 26.6 | 27.2 | 26.2 | 27.3 | 28.5 | 29.6 |
| LDLr+/− | T2 | 25.0 | 27.0 | 26.4 | 27.8 | 28.1 | 27.4 | 28.4 | 29.1 | 29.6 |
| LDLr+/− | T2 | 24.0 | 26.0 | 25.0 | 26.1 | 26.3 | 25.8 | 26.6 | 26.7 | 28.2 |
| LDLr+/− | T2 | 25.0 | 28.0 | 26.6 | 282 | 28.8 | 27.3 | 27.9 | 27.9 | 29.0 |
| | Ave | 25.2 | 27.2 | 26.7 | 28.1 | 28.6 | 27.6 | 28.5 | 29.2 | 30.0 |
| | Stdev | 1.60 | 1.47 | 1.70 | 1.69 | 1.82 | 1.68 | 1.69 | 2.05 | 1.60 |

TABLE 4
Body weight of Mice on High Dose T2

| | | | 1 Wk | | | | | |
|---|---|---|---|---|---|---|---|---|
| Genotype | Treatment | Baseline | Diet | Day 1 | Day 2 | Day 3 | Day 4 | Day 5 |
| C57 | Vehicle | 28.0 | 31.0 | 32.4 | 32.5 | 32.6 | 31.1 | 31.5 |
| C57 | Vehicle | 26.0 | 31.0 | 32.1 | 32.0 | 32.1 | 30.9 | 31.3 |
| C57 | Vehicle | 25.0 | 28.0 | 30.2 | 30.3 | 30.3 | 29.4 | 29.3 |
| C57 | Vehicle | 25.0 | 28.0 | — | — | — | — | — |
| C57 | Vehicle | 25.0 | 28.0 | 28.3 | 28.6 | 28.5 | 27.8 | 28.2 |
| C57 | Vehicle | 23.0 | 25.0 | 25.4 | 25.4 | 25.4 | 25.2 | 24.8 |
| | Ave | 25.3 | 28.5 | 29.7 | 29.8 | 29.8 | 28.9 | 29.0 |
| | Stdev | 1.63 | 2.26 | 2.90 | 2.88 | 2.93 | 2.45 | 2.73 |
| C57 | T2 | 26.0 | 27.0 | 29.6 | 29.5 | 29.8 | 29.1 | 29.7 |
| C57 | T2 | 24.0 | 28.0 | 29.6 | 30.1 | 30.0 | 28.7 | 29.1 |
| C57 | T2 | 23.0 | 24.0 | 24.9 | 25.8 | 25.1 | 23.4 | 24.1 |
| C57 | T2 | 24.0 | 28.0 | 29.8 | 29.7 | 29.9 | 28.8 | 28.5 |
| C57 | T2 | 24.0 | 24.0 | — | — | — | — | — |
| C57 | T2 | 23.0 | 26.0 | 26.7 | 27.1 | 27.2 | 27 | 27.3 |
| | Ave | 24.0 | 26.2 | 28.1 | 28.4 | 28.4 | 27.4 | 27.7 |
| | Stdev | 1.10 | 1.83 | 2.21 | 1.89 | 2.19 | 2.38 | 2.22 |
| LDLr0 | Vehicle | 28.0 | 30.0 | 32.4 | 31.8 | 32.1 | 31.2 | 31.8 |
| LDLr0 | Vehicle | 26.0 | 28.0 | 29.8 | 29.3 | 29.3 | 29.0 | 29.3 |
| LDLr0 | Vehicle | 23.0 | 25.0 | 23.6 | 23.7 | 23.5 | 23.9 | 23.8 |
| | Ave | 25.7 | 27.7 | 28.6 | 28.3 | 28.3 | 28.0 | 28.3 |
| | Stdev | 2.52 | 2.52 | 4.52 | 4.15 | 4.39 | 3.74 | 4.09 |
| LDLr0 | T2 | 22.0 | 24.0 | — | — | — | — | — |
| LDLr0 | T2 | 20.0 | 23.0 | 23.9 | 23.1 | 22.8 | 23.5 | 23.1 |
| LDLr0 | T2 | 22.0 | 23.0 | — | — | — | — | — |
| LDLr0 | T2 | 21.0 | 23.0 | 27.4 | 24.8 | 24.9 | 24.8 | 25.3 |
| | Ave | 21.3 | 23.3 | 25.7 | 24.0 | 23.9 | 24.2 | 24.2 |
| | Stdev | 0.96 | 0.50 | 2.47 | 1.20 | 1.48 | 0.92 | 1.56 |
| LDLr+/− | Vehicle | 23.0 | 24.0 | 24.9 | 24.7 | 25.4 | 23.9 | 24.4 |
| LDLr+/− | Vehicle | 24.0 | 25.0 | 25.5 | 25.4 | 25.6 | 24.4 | 24.7 |
| LDLr+/− | Vehicle | 24.0 | 26.0 | 28.5 | 28.6 | 27.9 | 27.0 | 26.7 |
| LDLr+/− | Vehicle | 25.0 | 26.0 | 27.0 | 26.9 | 26.6 | 25.9 | 26.3 |
| LDLr+/− | Vehicle | 28.0 | 32.0 | 29.6 | 29.8 | 30.0 | 27.7 | 26.0 |
| LDLr+/− | Vehicle | 27.0 | 31.0 | — | — | — | — | — |
| | Ave | 25.2 | 27.3 | 27.1 | 27.1 | 27.1 | 25.8 | 25.6 |
| | Stdev | 1.94 | 3.33 | 1.98 | 2.13 | 1.90 | 1.63 | 1.01 |
| LDLr+/− | T2 | 27.0 | 28.0 | 32.8 | 33.3 | 33.1 | 32.1 | 32.3 |
| LDLr+/− | T2 | 27.0 | 29.0 | 31.8 | 34.5 | 33.9 | 33.0 | 32.9 |
| LDLr+/− | T2 | 23.0 | 25.0 | 29.8 | 29.8 | 29.8 | 29.0 | 28.9 |
| LDLr+/− | T2 | 25.0 | 27.0 | 31.4 | 31.0 | 30.9 | 30.1 | 30.4 |
| LDLr+/− | T2 | 24.0 | 26.0 | 29.2 | 29.1 | 29.3 | 27.9 | 27.6 |
| LDLr+/− | T2 | 25.0 | 28.0 | 29.6 | 29.9 | 29.8 | 26.8 | 26.3 |
| | Ave | 25.2 | 27.2 | 30.8 | 31.3 | 31.1 | 29.8 | 29.7 |
| | Stdev | 1.60 | 1.47 | 1.44 | 2.16 | 1.92 | 2.40 | 2.61 |

TABLE 5
Cholesterol

| | Con | T2 | Con | T2 | Vehicle | T2 | Vehicle | T2 |
|---|---|---|---|---|---|---|---|---|
| | Before Diet | | 1 Week Diet | | 1 Week Low Dose | | 1 Week High Dose | |
| C57 | 92.072 | 101.333 | 142.251 | 178.120 | 165.859 | 104.366 | 195.259 | 75.711 |
| C57 | 90.013 | 118.313 | 178.784 | 204.690 | 211.429 | 114.798 | 202.026 | 62.741 |
| C57 | 103.906 | 97.217 | 158.857 | 103.061 | 195.507 | 137.858 | 194.695 | 70.636 |
| C57 | 97.732 | 104.421 | 132.952 | 163.507 | — | 96.130 | — | 41.312 |
| C57 | 91.043 | 102.877 | 126.309 | 138.265 | 154.878 | — | 184.545 | — |
| C57 | 93.101 | 94.13 | 115.68 | 1141.587 | 182.330 | 114.249 | 151.838 | 53.718 |
| Ave | 94.6 | 103.0 | 142.5 | 154.9 | 182.0 | 113.5 | 185.7 | 60.8 |
| Stdev | 5.27 | 8.38 | 23.05 | 35.31 | 22.61 | 15.66 | 19.92 | 13.72 |
| P-value | | | | | | 0.0005278 | | 0.0000029 |
| LDLr0 | 234.085 | 200.64 | 1381.710 | 994.950 | 1562.256 | — | 2146.77 | — |
| LDLr0 | 230.483 | 183.66 | 1461.340 | 1432.900 | 1644.582 | 1063.848 | 1629.59 | 303.529 |
| LDLr0 | 222.765 | 179.029 | 1194.020 | 1142.830 | 1400.940 | 593.252 | 891.684 | — |
| LDLr0 | | 168.224 | | 674.308 | | 813.532 | | 337.364 |
| Ave | 229.1 | 182.9 | 1345.7 | 1061.2 | 1535.9 | 823.5 | 1556.0 | 320.4 |
| Stdev | 5.78 | 13.49 | 137.25 | 315.65 | 123.94 | 235.46 | 630.77 | 23.92 |
| P-value | | | | | | 0.0097545 | | 0.0841325 |
| LDLr+/− | 117.799 | 117.284 | 214.653 | 188.748 | 245.470 | 185.075 | 264.62 | 84.169 |
| LDLr+/− | 115.74 | 129.119 | 201.368 | 286.391 | 244.921 | 229.548 | 240.936 | 99.395 |
| LDLr+/− | 114.197 | 136.837 | 180.113 | 241.223 | 263.589 | 196.605 | 247.702 | 103.342 |
| LDLr+/− | 126.546 | 134.264 | 229.267 | 259.822 | 285.550 | 160.917 | 224.582 | 102.778 |
| LDLr+/− | 149.186 | 118.313 | 275.763 | 204.690 | 239.431 | 172.447 | 464.243 | 66.124 |
| LDLr+/− | 132.206 | 145.069 | 219.967 | 299.012 | — | 188.370 | — | 107.29 |
| Ave | 125.9 | 130.1 | 220.2 | 246.6 | 255.8 | 188.8 | 288.4 | 93.8 |
| Stdev | 13.33 | 10.87 | 32.13 | 43.90 | 18.97 | 23.58 | 99.33 | 15.77 |
| P-value | | | | | | 0.0006384 | | 0.0010049 |
| | Before Diet | | 1 Week Diet | | 1 Week Low Dose | | 2 Week Low Dose | |
| LDLr0 | 175.960 | 149.702 | 662.800 | 727.240 | 845.940 | 611.760 | 939.265 | 996.905 |
| LDLr0 | 185.487 | 180.142 | 668.650 | 856.120 | 1438.580 | 750.720 | 1221.960 | 1035.325 |
| LDLr0 | 131.345 | 171.777 | 797.540 | 727.240 | 1028.640 | 813.250 | 1175.300 | 944.755 |
| LDLr0 | 228.708 | 194.549 | 1166.610 | 832.690 | 1626.150 | 1112.010 | 1960.260 | 988.670 |
| LDLr0 | 201.288 | 185.255 | 885.410 | 1055.300 | 1827.640 | 1167.590 | 1726.965 | 1092.965 |
| LDLr0 | 169.686 | 207.795 | 838.540 | 709.660 | 1174.540 | 438.070 | 1496.420 | 524.830 |
| LDLr0 | 190.831 | 177.354 | 1108.020 | 967.420 | 1612.250 | 1000.840 | 1847.730 | 1189.025 |
| LDLr0 | | 173.636 | | 1119.740 | | 1549.720 | | 1441.530 |
| Ave | 183.3 | 180.0 | 875.4 | 874.4 | 1364.8 | 930.5 | 1481.1 | 1026.8 |
| Stdev | 29.9597 | 17.0920 | 197.6447 | 157.7043 | 357.6818 | 352.0734 | 382.742 | 257.4105 |
| P-value | | | | | | 0.034183 | | 0.017125 |

TABLE 6
Blood Sugar

| | Before Diet | | 1 Week Diet | | 1 Week Low Dose | | 1 Week High Dose | |
|---|---|---|---|---|---|---|---|---|
| | Con | T2 | Con | T2 | Vehicle | T2 | Vehicle | T2 |
| C57 | 136 | 202 | 176 | 123 | 173 | 203 | 239 | 158 |
| C57 | 188 | 139 | 199 | 163 | 142 | 186 | 213 | 106 |
| C57 | 123 | 184 | 144 | 175 | 156 | 190 | 177 | 120 |
| C57 | 186 | 137 | 143 | 149 | — | 120 | — | 92 |
| C57 | 136 | 171 | 156 | 182 | 136 | — | 254 | — |
| C57 | 166 | 171 | 165 | 159 | 206 | 151 | 227 | 74 |
| Ave | 155.8 | 167.3 | 163.8 | 158.5 | 162.6 | 170.0 | 222.0 | 110.0 |
| Stdev | 27.97439 | 25.41391 | 21.32995 | 20.95471 | 28.13894 | 33.93376 | 29.3428 | 31.7805 |
| P-value | | | | | | 0.71715 | | 0.00041 |
| LDLr0 | 139 | 91 | 144 | 160 | 139 | — | 196 | — |
| LDLr0 | 150 | 111 | 181 | 111 | 170 | 143 | 164 | 87 |
| LDLr0 | 111 | 108 | 109 | 151 | 118 | 54 | 157 | — |
| LDLr0 | | 119 | | 141 | | 79 | | 101 |
| Ave | 133.3 | 107.3 | 144.7 | 140.8 | 142.3 | 92.0 | 172.3 | 94.0 |
| Stdev | 20.10804 | 11.78629 | 36.00463 | 21.2975 | 26.15977 | 45.90207 | 20.79263 | 9.899495 |
| P-value | | | | | | 0.174267 | | 0.017303 |
| LDLr+/− | 123 | 122 | 227 | 175 | 224 | 202 | 243 | 108 |
| LDLr+/− | 108 | 124 | 180 | 173 | 238 | 146 | 210 | 99 |
| LDLr+/− | 120 | 142 | 176 | 173 | 226 | 235 | 262 | 112 |
| LDLr+/− | 114 | 182 | 152 | 176 | 205 | 171 | 226 | 94 |
| LDLr+/− | 147 | 142 | 188 | 229 | 142 | 242 | 52 | 113 |
| LDLr+/− | 189 | 124 | 189 | 153 | — | 210 | — | 80 |
| Ave | 133.5 | 139.3 | 185.3 | 179.8 | 207.0 | 201.0 | 198.6 | 101.0 |
| Stdev | 30.28366 | 22.82688 | 24.42676 | 25.56886 | 38.20995 | 36.9973 | 84.20689 | 12.7122 |
| P-value | | | | | | 0.797769 | | 0.019686 |

These data show that T2 is more effective than statin drugs at lowering cholesterol and that T2 can decrease cholesterol via a mechanism that is independent of the low-density lipoprotein receptor (LDLr). In addition, intake of T2 can lower blood sugar levels relative to controls, which suggests that T2 may be an effective diabetes and metabolic syndrome treatment. Moreover, T2 may be combined with other thyroid hormones (e.g., T4 or T3), cholesterol lowering agents (e.g., statins), anti-diabetes agents, anti-hypersensives, anti-coagulants, anti-anginals, anti-arrhythmics, and/or vitamin and mineral compositions in order to augment the effects of known and yet to be discovered therapeutics and to help maintain healthy triglyceride and cholesterol levels, healthy weight, cardiovascular health, healthy sleep patterns, healthy mood, healthy skin, healthy nails, healthy endocrine function, healthy metabolism, healthy neuropsychiatric function, and a number of other markers of associated with general health and well-being. These data suggest that T2 may be effective for safely and effectively mitigating the effects of increased carbohydrate and/or fat intake and/or high cholesterol and hyperglycemia on normal human metabolic activity without disrupting endogenous thyroid function.

These are interesting and promising results because high cholesterol and hyperglycemia (i.e., diabetes mellitus) are known risk factors for cardiovascular disease. These results are also interesting because one active agent (i.e., T2) is conceivably able to address two known risk factors for cardiovascular disease.

Moreover, preliminary studies on T1AM action show that low doses (e.g., about 0.5 to 2 mg/kg body weight) of T1AM have similar effects as those effects observed for T2 on animals on a high fat diet. This suggests either that T2 and T1AM have similar modes of action or that one potential mechanism of action for T2 is through conversion to T1AM. As such, it is expected that administration of compounds that increase levels of T2, T1AM, and/or T0AM will have similar effects as those effects reported herein for T2.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A process for increasing T2, T1AM, and/or T0AM levels in a subject, comprising:
administering a fortified food, beverage, or dietary supplement composition to the subject, the fortified food or beverage composition including an effective amount of a compound of Formula I: 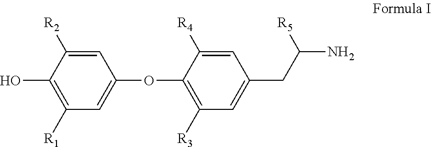
or a stereoisomer, prodrug, pharmaceutically acceptable salt, hydrate, solvate, acid salt hydrate, N-oxide, or isomorphic crystalline salt thereof; and
obtaining an effect of increasing T2, T1AM, and/or T0AM levels in the subject,
wherein R₁-R₄ are either I or H; and
wherein R₅ is either H or COOH;
with the proviso that the compound of Formula I is not 3,5,3′,5′-tetra-iodothyronine (“T4”) or 3,3′,5-triiodothyronine (“T3”), or a prodrug, pharmaceutically acceptable salt, hydrate, solvate, acid salt hydrate, N-oxide, or isomorphic crystalline salt of T4 or T3
wherein the effective amount of the compound of Formula I is effective for promoting healthy cholesterol levels, healthy triglyceride levels, healthy blood sugar levels, cardiovascular health, healthy sleep patterns, healthy mood, healthy skin, healthy nails, or healthy endocrine function and/or treating at least one of diabetes mellitus, fibromyalgia, sleep disorder, mood disorder, hyperglycemia, hypoglycemia, arthritis, physical or psychological condition caused by stress, or substance addiction in a human.

2. The process of claim 1, wherein the compound of Formula I is selected from the group consisting of T4AM, rT3, rT3AM, 3,3′-T2, 3,3′-T2AM, T2,3,5-T2AM, T1, T1AM, T0, T0AM, and combinations thereof.

3. The process of claim 1, wherein the compound of Formula I is T2 ((2S)-2-amino-3-[4-(4-hydroxyphenoxy)-3,5-diiodophenyl]propanoic acid) or a prodrug, pharmaceutically acceptable salt, hydrate, solvate, acid salt hydrate, N-oxide, or isomorphic crystalline salt thereof.

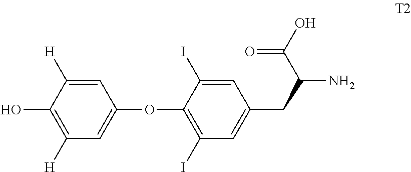

4. The process of claim 3, further comprising administering a compound selected from the group consisting of T4AM, rT3, rT3AM, 3,3′-T2, 3,3′-T2AM, 3,5-T2AM, T1, T1AM, T0, T0AM, and combinations thereof or a prodrug, pharmaceutically acceptable salt, hydrate, solvate, acid salt hydrate, N-oxide, or isomorphic crystalline salt thereof.

5. The process of claim 1, wherein the effective amount of the compound of Formula I comprises a daily dosage of between about 1 mcg and about 5000 mg.

6. The process of claim 1, wherein the effective amount of the compound of Formula I comprises a daily dosage of between about 10 mcg and about 1000 mg.

7. The process of claim 1, wherein the effective amount of the compound of Formula I comprises a daily dosage of between about 50 mcg and about 500 mg.

8. The process of claim 1, wherein the fortified food or beverage composition includes about 0.01 wt % to about 99.9 wt % of the compound of Formula I.

9. The process of claim 1, wherein the fortified food or beverage composition includes about 0.1 wt % to about 60 wt % of the compound of Formula I.

10. The process of claim 1, wherein the fortified food or beverage composition includes about 1 wt % to about 50 wt % of the compound of Formula I.

11. A process, comprising:
administering to a human a daily dosage ranging from about 1 mcg to about 5000 mg of a compound of Formula I or a stereoisomer, prodrug, pharmaceutically acceptable salt, hydrate, solvate, acid salt hydrate, N-oxide, or isomorphic crystalline salt thereof: 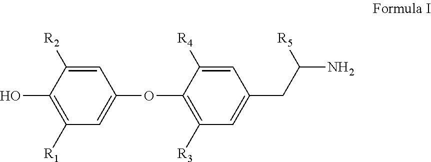
wherein:
R₁-R₄ are either I or H and R₅ is either H or COOH;
with the proviso that the compound of Formula I is not 3,5,3′,5′-tetra-iodothyronine (“T4”) or 3,3′,5-triiodothyronine (“T3”), or a prodrug, pharmaceutically acceptable salt, hydrate, solvate, acid salt hydrate, N-oxide, or isomorphic crystalline salt of T4 or T3;
obtaining an effect of promoting at least one of healthy cholesterol levels, healthy triglyceride levels, healthy blood sugar levels, cardiovascular health, healthy sleep patterns, healthy mood, healthy skin, healthy nails, or healthy endocrine function and/or treating at least one of diabetes mellitus, fibromyalgia, sleep disorder, mood disorder, hyperglycemia, hypoglycemia, arthritis, physical or psychological condition caused by stress, or substance addiction.

12. The process of claim 11, wherein the compound of Formula I is T2 ((2S)-2-amino-3-[4-(4-hydroxyphenoxy)-3,5-diiodophenyl]propanoic acid) or a prodrug, pharmaceutically acceptable salt, hydrate, solvate, acid salt hydrate, N-oxide, or isomorphic crystalline salt thereof.

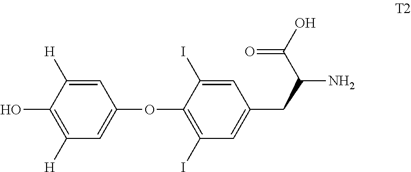

13. The process of claim 11, wherein the daily dosage of the compound of Formula I is at least about 5 mcg.

14. The process of claim 11, wherein the daily dosage of the compound of Formula I is at least about 100 mcg.

15. The process of claim 11, wherein the daily dosage of the compound of Formula I is at least about 1000 mcg.

16. The process of claim 11, wherein the daily dosage of the compound of Formula I is at least about 2000 mcg.
